(19) United States
(12) Patent Application Publication
(10) Pub. No.: US 2025/0303412 A1
(43) Pub. Date: Oct. 2, 2025

(54) APPARATUS, SYSTEM AND METHOD FOR MONITORING PATIENTS FOR INTERNAL INFECTION

(71) Applicants: Allison Lily Garrard, West Lafayette, IN (US); Iris Charlene Layadi, West Lafayette, IN (US); Pei-Lun Chen, West Lafayette, IN (US); Dhruv R. Patel, West Lafayette, IN (US); Shravan K. Ranganathan, West Lafayette, IN (US)

(72) Inventors: Allison Lily Garrard, West Lafayette, IN (US); Iris Charlene Layadi, West Lafayette, IN (US); Pei-Lun Chen, West Lafayette, IN (US); Dhruv R. Patel, West Lafayette, IN (US); Shravan K. Ranganathan, West Lafayette, IN (US)

(21) Appl. No.: 19/086,768

(22) Filed: Mar. 21, 2025

Related U.S. Application Data
(60) Provisional application No. 63/568,625, filed on Mar. 22, 2024.

(51) Int. Cl. B01L 3/00; A61B 5/20

(52) U.S. Cl. CPC B01L 3/502715; A61B 5/201; B01L 3/5023; B01L 2200/04; B01L 2300/027; B01L 2300/069; B01L 2300/0825

(57) ABSTRACT

An apparatus, system and method device for monitoring for an internal infection, such as a urinary tract infection. Included may be: a dispensing unit capable of receiving a continuous feed of color-changing infection test strips, comprising two compartments, one serving as a pass through for unused ones of the test strips, and the other configured to receive a used one of the test strips, and at least two gears capable of urging the continuous feed of the test strips from the unused compartment to the used compartment; a connection module capable of receiving a urine feed from a patient and having a window facing the dispensing unit; and a sensing module having an optical path facing the window and a urine reservoir for interfacing with the window to receive urine and including slits on either side thereof to feed the used ones of the test strips to the used compartment.

### CROSS-REFERENCE TO RELATED APPLICATIONS

This application claims priority to U.S. Provisional Patent Application No. 63/568,625, filed Mar. 22, 2024, which is incorporated herein by reference in its entirety.

### BACKGROUND OF THE DISCLOSURE

### Field of the Disclosure

The disclosure is directed to healthcare, and, more particularly, includes an apparatus, system and method for monitoring patients for internal infection.

### Description of the Background

The lack of early detection and monitoring tools for catheter-associated urinary tract infections (CAUTIs) highlights an evident need for a clinical solution. In the United States alone, CAUTIs represent a significant healthcare challenge, affecting over 560,000 patients annually and constituting more than 40% of hospital-acquired infections. As the predominant healthcare-acquired disease, CAUTI leads to increased patient morbidity, length of hospital stays, and associated healthcare costs, making the early and accurate detection of CAUTIs essential.

The over-reliance of current diagnostic procedures on symptom monitoring often causes diagnostic and treatment delays, subsequently increasing the risk of developing secondary complications. This issue is exacerbated in resource-limited care facilities and amongst the elderly patient population, who frequently present with ambiguous symptoms.

Currently, CAUTI is detected definitively via urine cultures and urinalysis tests, which are typically reactive and take days to produce results. This lag in detection significantly compromises patient outcomes, especially when executed after symptom onset. Despite advances in this specialty area, a fully integrated, automated, and real-time monitoring solution has been absent from the market.

Thus, the known art lacks devices that cater to early CAUTI detection as a supplement to definitive laboratory urinalysis and urine cultures. The current standard of care is therefore too heavily dependent on clinical monitoring, which is prone to diagnostic delays and the challenges associated with over- and under-treatment.

The foregoing disadvantages lead to increased patient discomfort and mortality, amplified healthcare expenditures, and extended hospital stays, presenting a significant burden on the healthcare system. As a result, the need exists for a a continuous monitoring, point-of-care testing (POCT) apparatus, system and method to provide rapid onsite evaluation of early CAUTI indicators, facilitating preemptive diagnosis and intervention.

### SUMMARY OF THE DISCLOSURE

The disclosed system integrates three different subcomponents, namely—1) a UTI test strip dispenser that is attached to 2) a connection module providing a pathway for urine flow, across from 3) an optical sensor that alerts medical staff upon detection of a color change on the test strips. Full implementation of the system is intuitive and can be easily integrated into existing medical workflows, providing a method for immediate analysis of urinary biomarkers (leukocyte) and streamlining the monitoring process.

The disclosure is targeted towards healthcare facilities and providers who utilize urinary catheters. With the disclosed embodiments, end-users may efficiently and effectively implement infection control measures to enhance patient care and reduce the incidence of CAUTIs. Further, the embodiments may be optimally integrated into current healthcare infrastructures.

The embodiments detect changes in leukocyte levels with precision. Sensitivity and specificity data associated with optical sensor performance further address the identified clinical need for timely CAUTI detection, offering a significant advantage over the known art that can be used to support standard diagnostic practices.

### BRIEF DESCRIPTION OF THE DRAWINGS

The foregoing and other aspects will become more apparent with reference to the detailed description below and the accompanying figures, which are included to provide an understanding of the disclosure and which constitute a part of the specification. In these drawings, like numerals may represent like elements, and:

FIGS. 1A-D illustrate aspects of the embodiments;

FIG. 2 illustrates aspects of the embodiments;

FIG. 3 illustrates aspects of the embodiments;

FIGS. 4A-C illustrate aspects of the embodiments;

FIG. 5 illustrates aspects of the embodiments;

FIGS. 6A-B illustrate aspects of the embodiments; and

FIG. 7 illustrates aspects of the embodiments.

### DETAILED DESCRIPTION OF THE DISCLOSURE

The figures and descriptions provided herein may have been simplified to illustrate aspects that are relevant for a clear understanding of the herein described devices, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are well known in the art, and because they do not facilitate a better understanding of the present disclosure, a discussion of such elements and operations is not provided herein. However, the present disclosure is deemed to inherently include all such elements, variations, and modifications to the described aspects that would be known to those of ordinary skill in the art.

Description is provided throughout so that this disclosure is sufficiently thorough and fully conveys the scope of the disclosed embodiments to those who are skilled in the art. Numerous specific details are set forth, such as examples of specific components, devices, and methods, to provide a thorough understanding of embodiments of the present disclosure. Nevertheless, it will be apparent to those skilled in the art that certain specific disclosed details need not be employed, and that embodiments may be embodied in different forms. As such, the embodiments described are exemplary in nature, and should not be construed to limit the scope of the disclosure.

Unless defined otherwise, all technical and scientific terms used herein have the same meaning as commonly understood by one of ordinary skill in the art to which this disclosure belongs. The terminology used herein is for the purpose of describing particular exemplary embodiments only and is not intended to be limiting.

Moreover, as used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. That is, terms such as “first,” “second,” and other numerical terms, when used herein, do not imply a sequence or order unless clearly indicated by the context. Thus, a first element, component, region, layer or section discussed below could be termed a second element, component, region, layer or section without departing from the teachings of the exemplary embodiments.

Further, the term “about,” as used herein when referring to a measurable value such as an amount, a temporal duration, and the like, is meant to encompass variations, such as of +20%, +10%, +5%, +1%, and +0.1% from the specified value, as such variations are appropriate. Similarly, throughout this disclosure, various aspects of the disclosure may be presented in a range format. It should be understood that a description in range format is merely for convenience and brevity, and thus should not be construed as an inflexible limitation on the scope of the disclosure. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, and so on, as well as individual numbers within that range, for example, 1, 2, 2.7, 3, 4, 5, 5.3, and 6. This applies regardless of the breadth of the stated range.

Processor-implemented modules and systems are disclosed herein that may provide access to and transformation of a plurality of types of non-transitory digital content, including but not limited to manufacturing plans and data streams, and the algorithms applied herein may track, deliver, manipulate, transform, transceive and report the accessed content. Described embodiments of these modules, apps, systems and methods are intended to be exemplary and not limiting.

An exemplary computing processing system for use in association with the embodiments, by way of non-limiting example, is capable of executing software, such as an operating system (OS), applications/apps, user interfaces, and/or one or more other computing algorithms, such as the algorithms, decisions, models, programs and subprograms discussed herein. The operation of the exemplary processing system is controlled primarily by non-transitory computer readable instructions/code, such as instructions stored in a computer readable storage medium, such as hard disk drive (HDD), optical disk, solid state drive, Random Access Memory (RAM), a flash memory, or the like. Such instructions may be executed within the central processing unit (CPU) to cause the system to perform the disclosed operations. In many known computer servers, workstations, mobile devices, personal computers, and the like, the CPU is implemented in an integrated circuit called a processor.

It is appreciated that, although the exemplary processing system may comprise a single CPU, such description is merely illustrative, as the processing system may comprise a plurality of CPUs. As such, the disclosed system may exploit the resources of remote CPUs through a communications network or some other data communications means.

In operation, CPU fetches, decodes, and executes the instructions from the computer readable storage medium. Information, such as the computer instructions and other computer readable data, is transferred between components of the computing system via the system's main data-transfer path.

In addition, the processing system may contain a peripheral communications controller and bus, which is responsible for communicating instructions from CPU to, and/or receiving data from, peripherals as discussed herein throughout. An example of a peripheral bus is the Peripheral Component Interconnect bus that is well known in the pertinent art.

An operator display/graphical user interface (GUI) may be used to display visual output and/or presentation data generated by or at the request of processing system, such as responsive to operation of the aforementioned computing programs/applications. Such visual output may include text, graphics, animated graphics, and/or video, for example.

Further, the processing system may contain a network adapter which may be used to couple to the aforementioned external communication network, which may include or provide access to the Internet, an intranet, an extranet, or the like. Communications network may provide access for processing system with means of communicating and transferring software and information electronically. Network adaptor may communicate to and from the network using any available wired or wireless technologies. Such technologies may include, by way of non-limiting example, wired Ethernet or fiber optic connections, cellular WAN infrastructures such as 3G, 4G/LTE, or 5G networks, Wi-Fi, Bluetooth®, Bluetooth® Low Energy (BLE), or Zigbee® links, infrared connections, or the like.

Currently, no market solutions exist that address the time gap between CAUTI onset early CAUTI detection to provide expedient treatment. The extended use of urinary catheters, sometimes lasting up to three months, remains a concern as longer dwell times often provide an ideal environment for pathogens to enter, which leads to infections.

To facilitate timely detection of CAUTI, the embodiments integrate with existing medical workflows without compromising standard urine testing procedures. The embodiments provide immediate or near-immediate visual signal upon detection of CAUTI indicators in the urine.

The embodiments reduce the average hospital length of stay due to CAUTIs, and exhibit higher specificity and sensitivity rates compared to existing at-home UTI testing methods. The embodiments experience a false positive rate of less than 10%, and a false negative rate of less than 5%.

The disclosure integrates a novel mechanism for dispensing UTI test strips in an automated manner, with an associated optical sensing technology that allows for high accuracy, specificity, and sensitivity in detecting color changes during test strip analysis. There is a specified time range for monitoring color changes, ensuring regular checks on patients. This prevents saturation issues, ensuring the method remains functional for the duration between a patient's average frequency of urination throughout the day.

The dimensions of the embodiments are parametrized to be smaller than the width and length of the drainage bag, ensuring a compact and convenient setup. It shares a similar weight to that of a catheter and follows a vertical catheter-drainage tube connection to prevent interruptions in the urine flow. The lifespan of this device aligns with the typical shelf life of catheters, drainage tubes, and bags.

The embodiments may include three main subcomponents, particularly the test strip dispenser, a connection module, and optical sensing unit. The disclosure may use pre-validated, commercially available UTI test strips, which are loaded into the test strip dispenser as one continuous strip.

Increases in leukocyte level within the patients' urine will be captured by the UTI test strip, producing a color change of the strip's reagent pads. These test strips are passed via the dispenser for the color change sensing, and are also be fed into the connection module attached to the dispenser. The connection module serves as a conduit through which the patients' urine will pass and interface directly with the reagent pads. Located along the drainage tubing pathway, the connection module displays the reagent pad to the built-in optical sensor, so that any color changes on the surface of the reagent pad are promptly detected. This detection may be signaled for electronic display on a medical monitoring system.

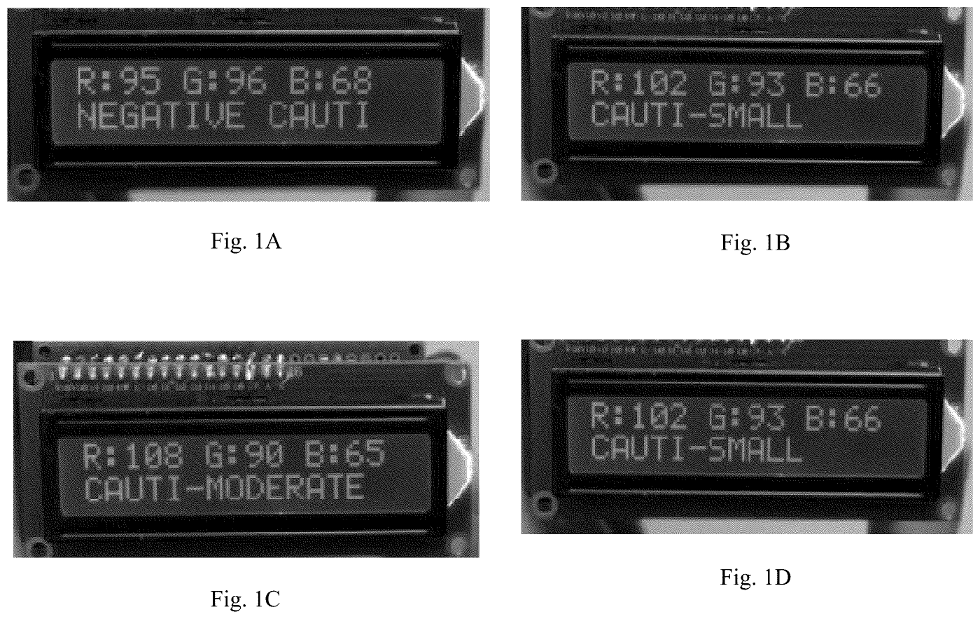

Although the skilled artisan will appreciate that various optical sensors may be used, an exemplary optical sensor may use RGB analysis to capture variations in color on the leukocyte reagent pad. It may be capable of detecting multiple, such as three, different levels of CAUTI. For example, detected may be levels corresponding to small, moderate, and large amounts of leukocyte. The automated interpretation of the optical sensor's RGB readouts may be displayed to the user, such as on an app graphical user interface, on an attached LCD screen, or the like, by way of non-limiting example. An exemplary automated indication may be as illustrated with respect to FIGS. 1A-D.

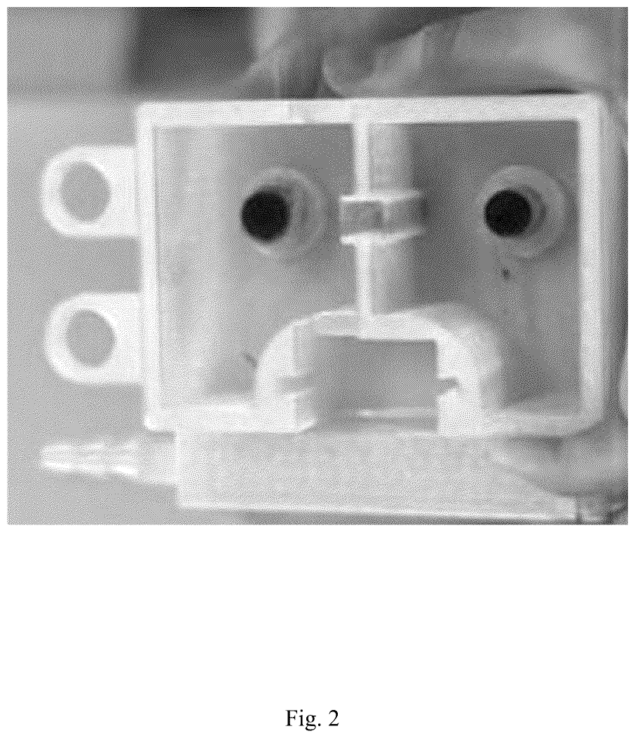

The disclosed dispensing device may be split into two chambers, as illustrated in FIG. 2. One chamber may house the clean roll of test strip reagent pads, and the other may store the used reagent pads, i.e., the waste.

Accordingly, the roll of reagent pads may be scrolled into the clean chamber, such as the lumen of the connection module, then into contact with the urine for reading by the optical sensor, and then into the waste chamber. The reagent pad roll may be made up of a clear cellophane backing, with each individual reagent pad attached a specific distance apart to account for the length between the openings for the clean and dirty chambers.

By way of example, the strips may be standard FDA-approved leukocyte test strips, with reagent pads mounted on a medical-grade polyethylene backing. These are spaced to correspond with the knob rotation and gear diameter, providing a new strip of reagent pads with every revolution. The strips may be laminated to secure the leukocyte reagent pads, then cut to fit the dispenser's dimensions. This layout may hold, for example, 24 test squares, which may be sufficient for a particular timeframe, such as a three-day period, assuming urinary frequency of every three hours.

The roll of test strip reagent pads may be rolled up around the prongs in the clean and waste compartments. Gears may then be used to feed the strip through the connection module, where urine passes through. Within the connection module and the test strip dispenser, slits allow for the dispensing and feeding of the test strips. Slits may be angled and rectangular, by way of example.

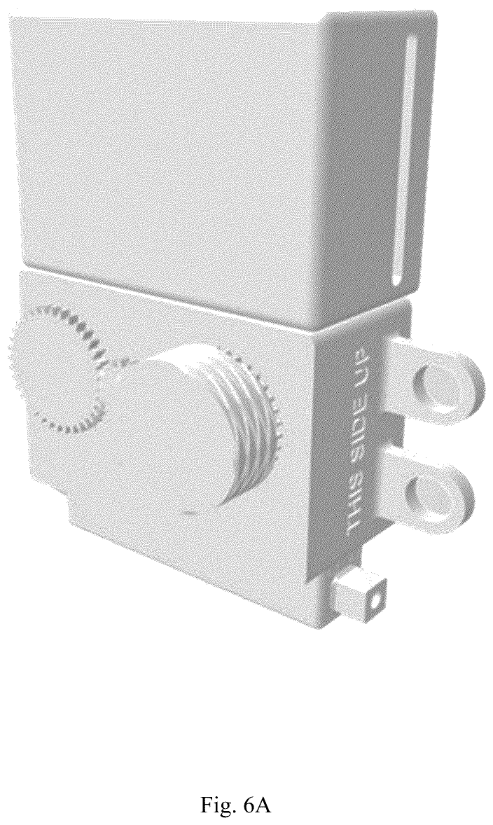

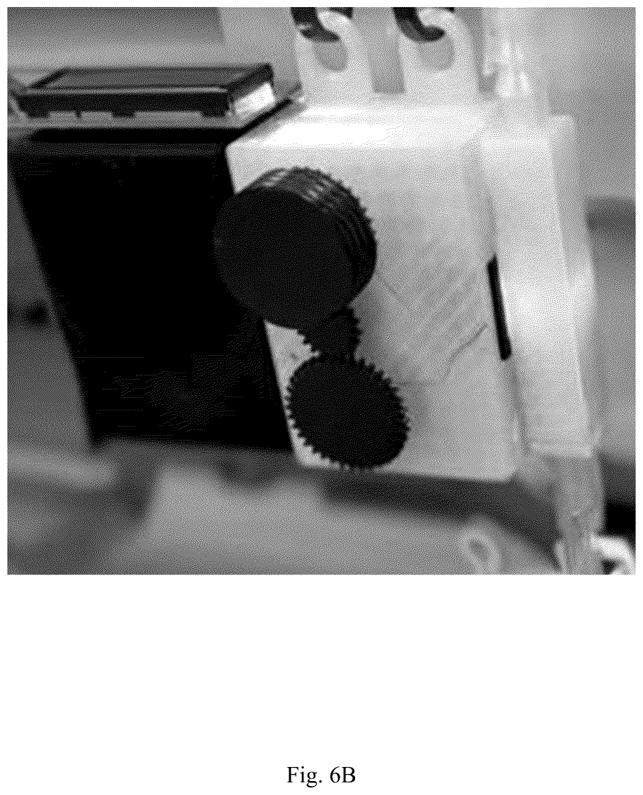

More specifically, the strips may be mounted on two cylinders aligned with the dispenser's gears. This enables the dispensing of a strip by simply rotating the knob on the exterior, which in turn activates the gear system. This action feeds a new strip through the slit, positioning it precisely for the sensor. Gear turning may be done manually or automatically, such as by rotation of the aforementioned knobs. The knobs may be sized and designed so as ensure easy and smooth one-handed operation of the gears in manual embodiments. An exemplary embodiment of the unit having operational manual knob is shown in FIGS. 6A and 6B.

More particularly, in FIG. 6A, the dispensing unit is at center with the knobs, and the connection module is at the bottom of the unit. In FIG. 6B, the dispensing unit, with knobs, is again at the center, and the connection module at the right thereof.

To prevent leakage and contamination between the clean and waste compartments within the dispenser, a medical-grade polyurethane gasket-like seal may be used to cover the slits. This material allows the test strips to be rotated out, but prevents any urine to enter back out of the connection module and into the dispenser. Similarly, to prevent urine from coming into contact with the optical sensor and other electrical components, a flexible, transparent low-density polyethylene cover may be utilized.

Operation involves rotating the knob to feed in a new test strip into position for exposure to and saturation by urine during each micturition event. When the strip reacts to the presence of leukocytes in urine, the reagent pads will change color, which will then be captured by the sensor and displayed digitally. After use, the strip is automatically moved to the waste compartment, ensuring sterile disposal. This system supports consistent use for in-patient monitoring whilst leveraging the benefits of current at-home UTI detection methods.

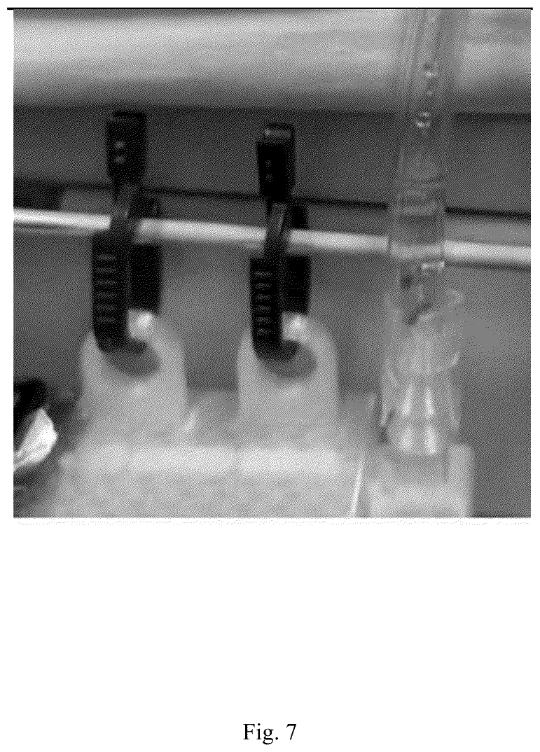

The housing may attach to the guardrail of the patients' bed, as recommended by clinical and nursing staff. For example and as also shown in FIG. 2, loops on the upper side of the dispenser may provide an easy method for attachment, and for portability purposes, while also keeping the housing out of the way of the patient. By way of example, FIG. 7 illustrates attachment of the unit to a bedside using the two loops provided, as well as a urine line running through the connection module next to the attachment loops. Moreover, this location will ensure that no backflow or blockage will occur throughout the length of the drainage tubing, which is important for infection control.

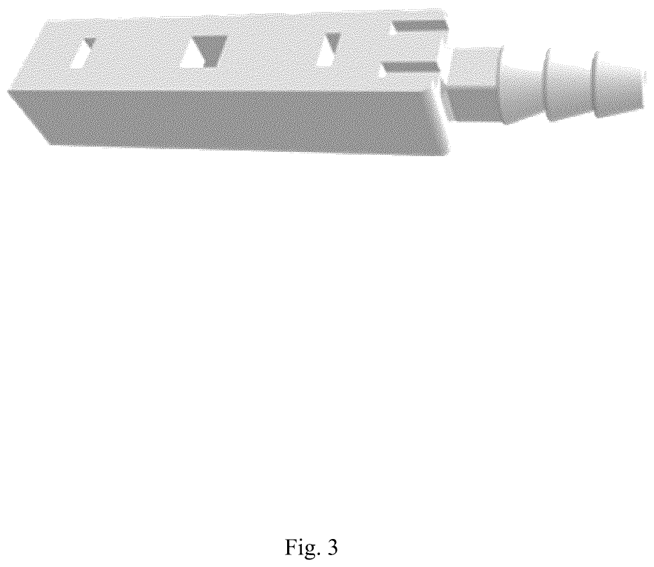

The connection module, as shown in FIG. 3, may include the mechanical capabilities to connect to existing foley catheter tubing and/or a urine bag. The connection module includes a connection to the UTI test strip dispenser, and allows the strip to be fed in and out of the lumen created by the connection module.

More specifically, the connection module connects and is in the flow between the catheter and the urine bag and includes the integrated UTI test strip dispenser/hosing. Urine flows from the catheter into the connector, where it contacts the test strip positioned within the module's flow path. A precision gear system within the dispenser may facilitate the movement of the test strip into the flow of urine.

This connection module may be universally compatible with various catheter and urine bag systems, eliminating the need for specialized manufacturing processes and thus reducing production costs. The connection module may be positioned above the urine bag to shorten the length of the urine bag tubing, which aids in supporting both the bag and the module. The top of the module may include a three-tiered adapter for secure attachment to the drainage tubing, designed to prevent leaks. The bottom of the module may comprise a streamlined design to ensure a direct flow into the urine bag, preventing backflow and kinking along the drainage tubing pathway.

The optical sensor, such as is illustrated in the exemplary embodiment of FIG. 3, may be housed within a casing attached to the test strip dispenser. The optical sensor detects and allows for analysis of RGB colors on the UTI test strip reagent pads. This connection to suitable analysis capabilities may be wired or wireless, and/or, in whole or in part, the analysis capability may be part of the sensor, such as resident in its firmware. Further, the optical sensor and/or the analysis capabilities may be communicative with the display, such as that shown in FIGS. 1A-D.

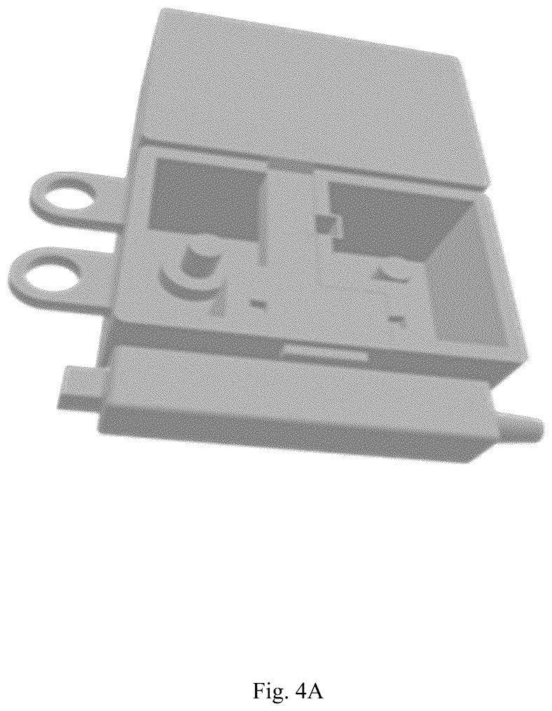

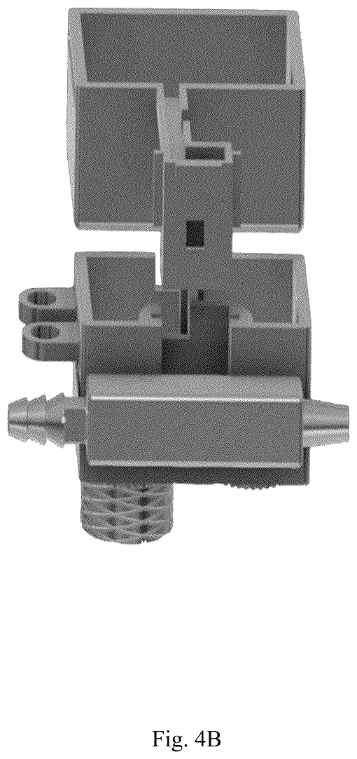

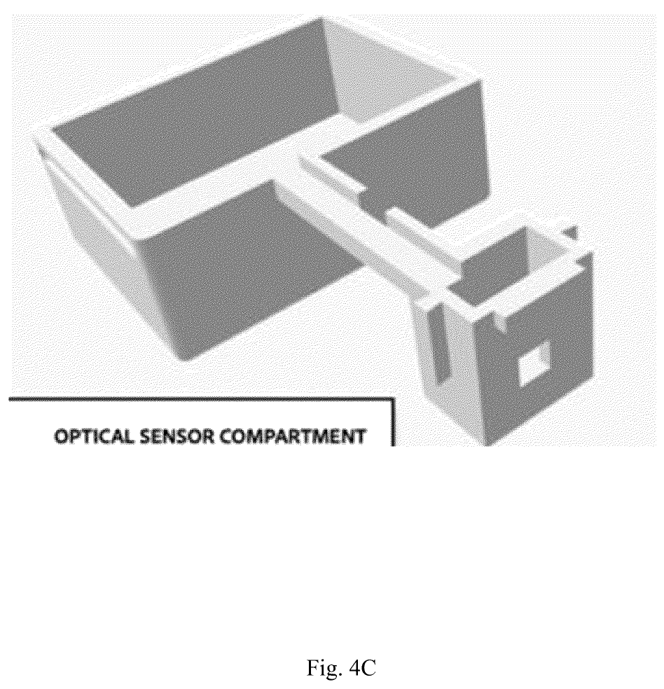

The full system, with the connection module positioned to pass urine onto the test strip, and expose the activated test strip to the optical sensor staged so as to view a strip section once it is exposed to urine in the connection module, is shown in FIG. 4B. Of note, the test strip will move through the unit from left to right as illustrated. That is, the clean test strip is within the left chamber, is exposed to the urine and is sensed, and then moves to the right chamber. Similarly, FIG. 4B illustrates the full unit, but with the top cover removed so as to more clearly illustrate the optical sensor “looking” at the test strip as the test strip is exposed to the urine flowing in the connection module.

FIG. 4B provides particular clarity in that is shows the sensor housing open, and illustrates the view path from the sensor to the test strip. The test strip would be extending between the slits on either side of the housing at the end of the view path, and the window that abuts the connection modules allows for entry of the urine through the window and onto the test strip. Once the test strip is wetted, it will change colors, which will be “seen” by the optical sensor as it views along the view path. This is made more clearly evident in the isometric view of FIG. 4C.

More particularly in relation to the optical sensor, white light interacts with the urine, the sample selectively absorbs or reflects specific wavelengths of light contingent upon its inherent color characteristics. The sensor employs photodetectors sensitive to diverse wavelengths, capturing the spectral information of the light that has interacted with the urine sample.

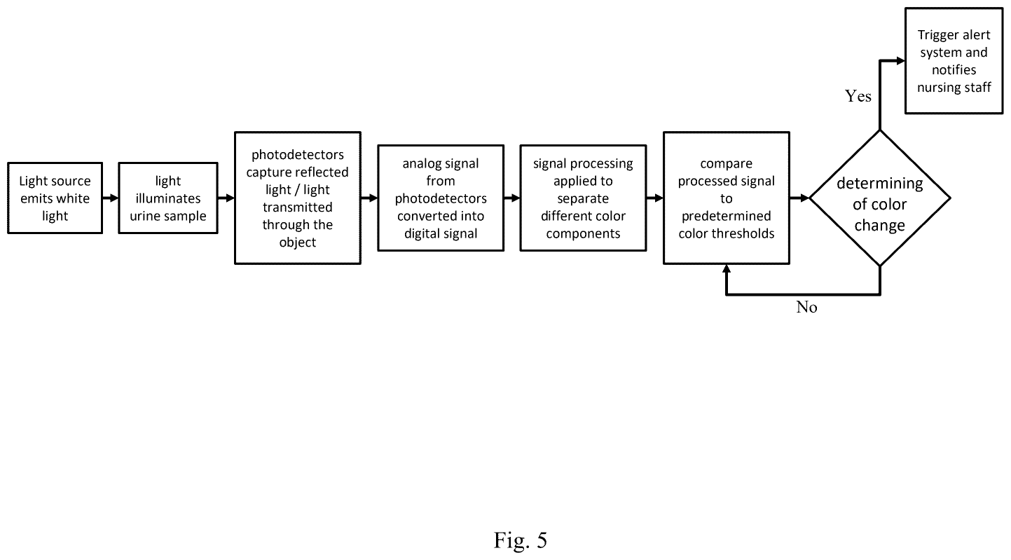

Subsequently, an analog-to-digital conversion process may translate this optical signal into a digital format for further analysis. Signal processing algorithms are then employed to extract and discern the spectral components representative of distinct colors within the urine sample. These spectral features are subsequently cross-referenced against predetermined color standards or thresholds stored in a computing memory. In the event of a substantial deviation from the established color reference values, the sensor registers a color change within the urine sample and generates an output or alert signal. The aforementioned process is further illustrated in relation to the flowchart in FIG. 5.

In relation specifically to the optical sensor and by way of non-limiting example, the optical sensor may be a TCS34725 optical sensor. An exemplary microcontroller utilized for an optical sensor setup may be the Arduino Nano.

Needless to say, the disclosed system and method may additionally include other elements. By way of example, the measuring unit may include power, such as via a power feed or a battery. Further included may be a communications card, such as for wired or wireless communication into and out from the unit. This communication may occur to a processor, which may itself communicate with an app, such as on a computer or a mobile device.

The disclosed solution thus directly integrates a test strip dispenser with an optical sensor, enabling continuous, real-time surveillance of patients' urine status and substantially narrowing the delay between infection onset and detection. The device differs from traditional urine cultures and symptomatic reporting by providing proactive, uninterrupted monitoring. Employing pre-validated FDA-approved test strips ensures reliability, while the optical sensor employs precise RGB analysis to analyze color changes that may occur on the test strips' reagent pads, indicating the presence and specific level of leukocytes, enhancing diagnostic efficiency and accuracy.

Of additional note, the housing of the test strip dispenser unit as well as the sensor compartment may be made from PLA, which is robust and withstands external trauma and chemical processing needed for sterilization. The optical sensor may be encased within a waterproof housing and may include a transparent polyethylene barrier between the sensor and the view path. The connection module may be fabricated using PET-G, and may also be printed, together with the other components and sub-components, via additive manufacturing for ease of assembly. The connection module may feature universal connectors to ensure compatibility with a range of Foley catheter and drainage tubing brands and sizes.

Thereby, the disclosure includes UTI test strips that can be fed via continuously from a novel dispensing design into a connection module, where it will become saturated with urine. The optical sensor component remains the same as our initial design, but is located across from the opening on the connection module that exposes the reagent pads on the test strip, so that any color change will be captured and signaled to the nursing staff. The disclosed apparatus, system and method thereby consistently monitors urinary biomarkers (leukocytes) indicative of infection.

In sum, the disclosed system and method provides a manual or automated test strip dispenser for the continuous monitoring of patients' urinary status. Included may be a rotary test strip dispenser, a catheter and drainage bag-compatible connection module, and an optical sensor that executes RGB analysis. The device may operate within the existing infrastructure of urinary catheterization in clinical settings.

The embodiments may be used in hospital environments, long-term care facilities, and medical clinics where urinary catheterization is a standard practice, by way of non-limiting example. The embodiments enable the preemptive detection of CAUTIs, thus mitigating patient risk and reducing the economic burden associated with late-stage infection management. The embodiments provide a complementary detection and monitoring tool to support standard diagnostic procedures, including urinalyses and urine cultures.

In the foregoing detailed description, it may be that various features are grouped together in individual embodiments for the purpose of brevity in the disclosure. This method of disclosure is not to be interpreted as reflecting an intention that any subsequently claimed embodiments require more features than are expressly recited.

Further, the descriptions of the disclosure are provided to enable any person skilled in the art to make or use the disclosed embodiments. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but rather is to be accorded the widest scope consistent with the principles and novel features claimed as follows.

What is claimed is:

1. A device for monitoring a patient for a urinary tract infection, comprising:
a dispensing unit capable of receiving a continuous feed of color-changing urinary tract infection test strips, the dispensing unit comprising:
two compartments, one of the two compartments serving as a pass through for unused ones of the test strips, and a second of the two compartments configured to receive a used one of the test strips; and
at least two gears capable of urging the continuous feed of the test strips from the unused compartment to the used compartment;
a connection module capable of receiving a urine feed from a patient and passing the urine feed to a urine bag, the connection module also having a window facing the dispensing unit; and
a sensing module having an optical path facing the window and, at a distal end of the optical path, a urine reservoir having a second window for interfacing with the window to receive urine, the urine reservoir further including slits on either side thereof, one of the slits configured to receive the continuous feed from the unused compartment, and an other of the slits configured to feed the used ones of the test strips to the used compartment;
wherein the sensing module optically senses a color of the test strip as it is in the presence of the urine feed within the urine reservoir, and wherein communication of the color to a communicative processor enables a recognition of the urinary tract infection.
